(19) United States
(12) Patent Application Publication
(10) Pub. No.: US 2003/0165568 A1
(43) Pub. Date: Sep. 4, 2003

(54) Stabilized steroidal suspension

(76) Inventors: Giuseppe Colombo, Milano (IT); Alessandro Martini, Milano (IT); Sharron Buttler, Vicksburg, MI (US)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/275,955

(22) PCT Filed: Apr. 25, 2001
(86) PCT No.: PCT/EP01/04642

(51) Int. Cl. A61K 9/14

(52) U.S. Cl. 424/486

(57) ABSTRACT

A pharmaceutical aqueous suspension formulation for parenteral administration having substantially stabilized pH, comprising a biologically active compound and a pH controlling effective concentration of a polyvinylpyrrolidone compound. Preferably the biologically active compound is a steroidal compound, for instance, exemestane, medroxyprogesterone acetate or estradiol cypionate or a combination of medroxyprogesterone acetate and estradiol cypionate.

### SUMMARY OF THE INVENTION

The present invention is in the field of galenic preparations. It concerns in particular a pharmaceutical aqueous suspension of a biologically active compound, e.g. a steroidal compound, having stabilized pH, particularly suitable for parenteral administration. The inventors of the present invention have found that the pH of a pharmaceutical aqueous suspension of a biologically active compound can be controlled by including a pH controlling effective concentration of a polyvinylpyrrolidone (PVP) compound thereto.

Moreover, when a pH controlling effective concentration of a PVP compound is used, PVP strengthens the buffering capacity of low concentrations of conventional buffering agents with a superadditive (synergistic) effect. In this way the use of conventional buffering agents in usual concentrations can be eliminated or limited, thus improving the re-suspendability and controlled flocculation of the pharmaceutical preparation.

### BACKGROUND OF THE INVENTION

A pharmaceutical suspension is a coarse dispersion in which insoluble solid particles are dispersed in a liquid medium.

Suspensions contribute to pharmacy and medicine by supplying insoluble and often distasteful substances in a form that is pleasant to the taste, by providing a suitable form for the application of dermatological materials to the skin and sometimes to the mucous membranes, and for the parenteral administration of insoluble drugs. Therefore pharmaceutical suspensions may be classified into three groups: orally administered mixtures, externally applied lotions and injectable preparations.

An acceptable suspension possesses certain desirable qualities, including the followings:

i) the suspended material should not settle rapidly;

ii) the particles that do settle to the bottom of the container must not form a hard cake but should be readily re-dispersed into a uniform mixture when the container is shaken;

iii) the suspension must not be too viscous to pour freely from the orifice of the bottle or to flow through a syringe needle.

It is important that the characteristics of the dispersed phase are chosen with care so to produce a suspension having optimum physical, chemical and pharmacological properties. Particle size distribution, specific surface area, inhibition of crystal growth, and changes in the polymorphic form are of special significance and the formulator must ensure that these and other properties do not change sufficiently during storage to adversely affect the performance of the suspensions with aging.

In the field of injectable preparations, aqueous suspensions of drugs, e.g. steroids, for parenteral administration have already been described in scientific and patent literature and have been known for a long time.

It is well known that one of the main difficulties in formulating steroids is the need to overcome their hydrophobicity, that significantly reduces the suspendability or re-suspendability of the active in aqueous media. Both wetting and suspending agents are needed in order to gain the proper formulation of the active compound such as the concomitant use of preservatives. This is described, for example, by Nash and coworkers in the U.S. Pat. No. 3,457,348, where non-ionic surfactants (such as polysorbates) and suspending agents (like polyethylenglycols) are the basic excipients to gain the proper stability of the formulation.

Sometimes, even in the presence of the proper suspending and wetting agents, the suspension is not stable for a long time, but it is necessary to form it just before the administration (while it is stored as lyophilized formulation). This is described, for example, in the case described by Geller and coworkers in the U.S. Pat. No. 5,002,940 and greatly impacts on the cost of the manufacturing process, since an additional freeze-drying process is mandatory.

Even if an improved physical stability of drug suspensions in water can be gained, as above mentioned, by the use of polyethylenglycols and non-ionic surfactants, some chemical stability issues, such as a relevant pH reduction, are likely to be faced during development.

In fact, for instance, both polyethylenglycols and polysorbates, when in solution, may undergo degradation, leading to the formation of acid species such as formic and acetic acid.

This fact necessarily causes the reduced shelf life of parenteral suspension, being the progressive acidification of the formulation linked to the impossibility to administer the formulation, e.g. by intramuscular or subcutaneous injection, without generating significant pain on patients (it is advisable that the pH value is maintained above 3 for administering a painless formulation).

This pH variation during storage can be minimized by appropriately buffering the to formulation. The most obvious approach, in order to maintain the pH within specific and predetermined limits, is the use of buffering agents, such as inorganic acid salts, in effective concentrations in order not only to exert but also to maintain their buffering capacity. An example of buffering agents commonly used in parenteral formulations and of their usual concentrations can be found in Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products, Marcel Dekker Inc.

The use of organic and inorganic acid salts as buffering agents offers to the formulator both advantages and disadvantages. In fact, if a careful control of pH of formulations could be gained, on the contrary, when suspension formulations are concerned, ionic species tend to destabilize the formulations with detrimental effects on their re-suspendability and on their controlled flocculation. This means that the use of inorganic acid salt based buffering systems into the formulations has to be minimized.

This fact is well known in the prior art about parenteral suspensions, as clearly stated by Nash (Parenteral Suspensions, Bulletin of Parenteral Drug Association, March-April 1972, Vol. 26, No. 2), “. . . indiscriminate use of salts and buffers is normally avoided, provided chemical stability is not a problem since changes in electrolyte concentration often have a profound effect on the absorbed surface charge of suspension particles . . . .”

### DESCRIPTION OF THE INVENTION

The inventors of the present invention have found that suitable concentrations of a PVP compound are able both to control the pH of a pharmaceutical aqueous suspension of a biologically active compound, in particular a steroidal compound, by minimizing its pH decrease and to strengthen the pH controlling capacity of lower and unusual concentrations of conventional buffering agents, with a superadditive (synergistic) effect.

A superadditive (synergistic) effect is a pH controlling effect that is greater than the one which is expected to be obtainable by summing up the experimentally verified pH controlling effects of the single agents.

While PVP is widely used in parenteral formulations as suspending agent, its unexpected property of pH controller in aqueous suspensions has never been disclosed before.

The conventional applications of PVPs in pharmaceuticals are here below briefly summarized.

The soluble grades of PVP possess a number of very useful properties for which they are widely used in pharmaceuticals.

Because of these properties, the products can perform different functions in different dosage forms.

Its excellent solubility in water and in other solvents used in pharmaceutical production is an advantage in almost all dosage forms. PVP is used in wet granulation in tablet production (where it works as a binder), in oral solutions, syrups and drops, in injectables and topical solutions and in film coating of tablets.

Its adhesive and binding power is particularly important in tableting (wet granulation, dry granulation, direct compression). This property is also useful in film coatings and adhesive gels.

Its film-forming properties are used in the film coating of tablets, in transdermal systems and in medicinal sprays.

Its affinity to hydrolytic and hydrophobic surfaces is particularly useful in the hydrophilization of a wide range of substances, ranging from hydrophobic tablet cores to permit sugar or film coating to medical plastics.

Its ability to form complexes with a large number of substances is also pharmaceutically useful. The complexes formed are almost always soluble and are chemically stable in an acid environment. This property can be used to increase the solubility of drugs in liquid dosage forms.

In solid dosage forms, the ability to form complexes is used to increase bioavailability. Sometimes complexation is also used for reducing the local toxicity of certain drugs. Its thickening properties are used in oral, topical liquid dosage forms, e.g. syrups and suspensions.

As said above, in spite of the number of conventional applications in pharmaceuticals, the pH controlling properties of PVP in parenteral aqueous suspensions are unexpected, new and innovative.

Accordingly one object of the present invention is to provide a pH substantially stabilized pharmaceutical aqueous suspension formulation for parenteral administration comprising a biologically active compound and a pH controlling effective concentration of a PVP compound.

Object of the invention is also the use of a PVP compound, in a pH controlling effective concentration, in the preparation of a pharmaceutical aqueous suspension formulation having substantially stabilized pH, for parenteral administration of a biologically active compound.

A further object is a method for preparing a substantially stabilized pH pharmaceutical aqueous suspension formulation for parenteral administration of a biologically active compound, characterized in that a pH controlling effective concentration of a PVP compound is added thereto.

The inventors have also found that PVP, besides exercising a pH controlling activity per se, also strengthens the pH controlling capacity of a conventional buffer with a (superadditive) synergistic effect. A superadditive (synergistic) effect is a pH controlling effect that is greater than the one which is expected by summing up the experimentally verified pH controlling effects of the single agents.

This means that lower and unusual concentrations of conventional buffering agents can be included into the formulations, without any risk of deteriorating the physico-technological quality of parenteral suspensions.

A further advantage is given by the fact that as no relevant concentrations of buffers are needed, the formulation has low or no buffering capacity per se and therefore, once administered the pH of the formulation will be easily adjusted to the physiological value by the buffering capacity of body fluids.

As stated above, the reduction in the quantity of conventional buffering agents, such as inorganic acid salts, improves the physical stability of the formulation, since ionic species tend to destabilize the formulations with detrimental effects on their re-suspendability and on their controlled flocculation.

A further object of the invention is therefore to provide a pH substantially stabilized pharmaceutical aqueous suspension formulation for parenteral administration comprising a biologically active compound, a buffering agent and a PVP compound in concentrations effective to produce a pH controlling superadditive effect. The present invention also provides the use of a PVP compound and a buffering agent in concentrations effective to produce a pH controlling superadditive effect, in the preparation of a pharmaceutical aqueous suspension formulation having substantially stabilized pH, for parenteral administration of a biologically active compound. The term “a buffering agent” is herein meant to comprise (unless otherwise specified) both a single buffering agent and a mixture of two or more thereof. The term “substantially pH stabilized” means that the pH of the formulation remains within acceptable limits for parenteral administration over the time, according to well known practice in the art. It also means that the pH of the formulations containing a PVP compound, or the combination of a PVP compound and a buffering agent in concentrations effective to provide a pH controlling super-additive effect, is maintained closer to the initial value than the pH of the “as is” formulation (i.e. the formulation without the PVP compound or the combination of the PVP compound and a buffering agent.

The pH range for the suspension formulation of the invention is from about pH 3.0 to about pH 8.0, preferably pH 3.5 to pH 7.5, and most preferably pH 4.0 to pH 7.0.

The pharmaceutical aqueous suspension, according to the invention, may in addition also include one or more surfactants, suspending agents and/or thickening agents.

Suitable surfactants are for instance phospholipids (e.g. lecithin), cationic surfactants (e.g. miristylgammapicolinium chloride), anionic-surfactants and non-ionic surfactants, preferably polysorbates (e.g. Polysorbate 80).

Suitable suspending agents are for instance polyethylenglycols, preferably polyethylenglycols having a molecular weight from about 300 to about 6000, (e.g. polyethylenglycol 3350 and polyethylenglycol 4000).

Suitable thickening or viscosity agents are for instance well known cellulose derivatives, (e.g. methylcellulose, sodium carboxymethylcellulose, hydroxyethylcellulose and hydroxypropylmethylcellulose) gelatin and acacia.

In addition, the formulations of the present invention may also include metal chelating agents, antioxidants or thiol-containing compounds and preservatives.

Suitable metal chelating agents are for instance ethylenediamine-tetracetic acid salts, (e.g. edetate disodium).

Suitable antioxidants are for instance ascorbic acid derivatives (e.g. ascorbic acid, erythorbic acid, sodium ascorbate) or thiol-derivatives (e.g. monothioglycerol, cystine, acetylcysteine, cysteine, dithioerythreitol, dithiotheitol, gluthathione and L-methionine, in particular L-methionine) tocopherols, butylated hydroxyanisole, butylated hydroxytoluene, sulfurous acid salts (e.g. acetone sodium bisulfite, sodium sulfite, sodium formaldehyde sulfoxylate, sodium thiosulfate, sodium sulphate, sodium bisulfite, and sodium metabisulfite), nordihydroguairaretic acid.

Suitable preservatives are for instance phenol, chlorobutanol, benzylalcohol, methyl parabens, propyl parabens, benzalkoniurn chloride and cetilpiridinium chloride, and preferably methyl and propyl parabens.

In addition, the formulations of the present inventions may also include tonicity adjusting agents. Suitable tonicity adjusting agents are for instance sodium chloride, sodium sulphate, dextrose, mannitol and glycerol.

The formulations of the present invention may also have a nitrogen blanket overlay on the headspace of the vial. Additionally, the formulations of the present invention may include purging the formulation with helium, argon, or nitrogen.

When the formulation of the invention, besides a PVP compound, contains also buffering agents, useful buffers include e.g. those derived from acetic, aconitic, citric, glutaric, lactic, malic, succinic, phosphate and carbonic acids, as known in the art. Typically employed is an alkali or alkaline earth salt of one of the aforementioned acids. Phosphate and citrate buffers, such as phosphoric acid or a pharmaceutically acceptable salt thereof, or citric acid or a pharmaceutically acceptable salt thereof, are preferred.

Sodium phosphate, or sodium citrate are the preferred buffering agents, with sodium phosphate being most preferred.

The pharmaceutical aqueous suspension according to the invention is e.g. for intramuscular, subcutaneous and intradermal administration, preferably for intramuscular and subcutaneous administration.

A biological active compound according to the invention is any compound which after administration to a mammal, including humans, provides a therapeutic effect.

Preferably it is a steroidal biologically active compound.

A steroidal biologically active compound according to the invention is the steroidal compound itself or, when appropriate, a pharmaceutically acceptable salt thereof as known in the art, e.g. medroxyprogesterone acetate, exemestane, estradiol cypionate, methylprednisolone acetate, oxabolone cypionate, clostebol acetate, testosterone cypionate; preferably medroxyprogesterone acetate, estradiol cypionate and exemestane, or a combination of two or more thereof according to the art.

Concentrations of medroxyprogesterone acetate in the formulation can range from about 1% w/v to about 40% w/v, preferably from about 3% w/v to about 30% w/v.

Concentrations of estradiol cypionate in the formulation can range from about 0.1% w/v to about 5% w/v, preferably from about 0.25% w/v to about 2.5% w/v.

When a combination of estradiol cypionate and medroxyprogesterone acetate is the active ingredient of the pharmaceutical preparation of the invention, the concentrations of such compounds present in the pharmaceutical preparation are substantially as here above.

Concentrations of exemestane in the formulation can range from about 1% w/v to about 25% w/v, preferably from about 5% w/v to about 20% w/v.

The steroidal biologically active compound is preferably in milled or micronized form according to the common practice.

Preferred PVP compounds according to the invention are those having a molecular weight from about 7000 to about 54000, for instance PVP K12, K17, K25 and K30, in particular K12 and K17, PVP K17 being the most preferred.

The main difference among the four above mentioned grades of PVP are their average molecular weights, that are expressed in terms of their K-values, in accordance with the European and US Pharmacopeias, or as Weight average (Mw), as clearly described by Buhler in ‘Polyvinylpyrrolidone for the pharmaceutical industry’, (1992) 34-38. Where the Mw of PVP K12 corresponds to an interval between 2000 and 3000, the Mw of PVP K17 corresponds to an interval between about 7,000 and about 11,000; PVP K25 and K30 correspond to higher Mw values in the intervals ranging between about 28,000-34,000 and about 44,000-54,000 respectively.

Even more surprisingly, the pH controlling capacity of PVP depends on the grade of polyvinylpyrrolidone used.

When a particular grade of PVP is used, namely K17, the effects on pH control are more evident. Accordingly the use of PVP K17 is a preferred feature of the invention. A pH controlling effective concentration of PVP, when used as a single pH controlling agent, may vary from about 0.1% w/v to about 10% w/v.

In particular the pH controlling effective concentration of PVP K17, when used as a single pH controlling agent, may vary preferably from about 0.2% w/v to about 5% w/v.

Thanks to the pH controlling properties of PVP and the superadditive pH controlling effect, which is obtainable by using PVP in combination with a conventional buffering agent, the usual buffering concentration of the latter can be reduced by about 50% to about 95%. Namely the concentration of the buffering agent can thus range from about 5% to about 50% of the usual buffering concentration thereof, preferably from about 5% to about 25%.

The usual concentrations of conventional buffering agents used in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products, Marcel Dekker Inc.

According to said literature, for instance the usual buffeting concentration for phosphoric acid salts range from about 0.8% to about 2.0% w/v or w/w. On the contrary, thanks to the newly found superadditive effect, the concentration of phosphoric acid salts according to the formulation of the invention are lower than 0.4% w/w or w/v, preferably lower than 0.2% w/w or w/v.

Re-suspendability and controlled flocculation of the pharmaceutical aqueous suspensions are thus improved.

The pH controlling activity of PVP alone or in combination with a conventional buffer is shown for instance by the following examples.

### EXAMPLE 1

The influence of polyvinylpyrrolidone (PVP) K17 as pH controlling agent in a typical vehicle composition suitable for sterile aqueous suspension (batch 13169/12-A) is shown in Table 1, where the measured pH values recovered in an accelerated stability study are shown.

The vehicle composition (methylparaben 0.2%, propylparaben 0.02%, sodium chloride 0.9%, PEG 4000 3%, Polysorbate 80 0.3%, sodium hydroxide q.s. to pH 6.5, WFI q.s to 100 ml. Where PVP is added, according to the scheme in Table 1, the pH is adjusted to 6.5 after adding PVP), has been used to fill a 20/26 mL clear type I siliconized glass vials, 20 mm diameter that has then been closed with an inert coated (Omniflex®) rubber stopper. 20 mL of vehicle has been put in each vial. The PVP K17 used is a Kollidon 17PF from BASF.

TABLE 1

| | | PVP K17 | PVP K17 |
|---|---|---|---|
| | As is | 0.5% | 2% |
| Time zero | 6.46 | 6.47 | 6.51 |
| 65° C. | | | |
| 10 days | 3.43 | 3.67 | 4.04 |
| 15 days | 3.16 | 3.37 | 3.69 |
| 1 month | 3.32 | 3.48 | 3.71 |
| 40° C. | | | |
| 3 months | 3.24 | 3.93 | 4.19 |
| 6 months | 3.15 | 3.27 | 3.42 |
| 25° C. | | | |
| 6 months | 4.93 | 5.60 | 5.51 |

It is evident that when PVP is present into the formulation, the reduction in pH value of the vehicle with aging is minimized.

### EXAMPLE 2

The influence of PVP K17 as pH controlling agent in a typical buffered vehicle composition suitable for sterile aqueous suspension is shown in Table 2, where the measured pH values recovered in an accelerated stability study are shown.

The vehicle composition (methylparaben 0.2%, propylparaben 0.02%, sodium chloride 0.9%, PEG 4000 3%, polysorbate 80 0.3%, sodium hydroxide q.s. to pH 6.5, WFI q.s 10 to 100 ml. Where PVP is added, according to the scheme in Table 2, the pH is adjusted to 6.5 after adding PVP), has been used to fill a 20/26 mL clear type I siliconized glass vials, 20 mm diameter that has then been closed with Omniflex® rubber stoppers. 20 mL of vehicle has been put in each vial. The PVP K 17 used is a Kollidon 17PF (BASF) and the PVP K 30 used is a Povidone USP Grade. The phosphate buffer 0.0066 M (approximately 0.1% w/v) has the following composition: Monobasic sodium phosphate hydrate 69.4 mg and dibasic sodium phosphate dodecahydrate 58.8 mg in 100 mL of vehicle; while the phosphate buffer 0.066 M (approximately 1% w /v) has the following composition: Monobasic sodium phosphate hydrate 694 mg and dibasic sodium phosphate dodecahydrate 588 mg in 100 mL of vehicle.

TABLE 2

| | 13169/12 - E | | | | 13169/12 - F | | | |
|---|---|---|---|---|---|---|---|---|
| | Phosphate pH 6.4 | | | | Phosphate pH 6.4 | | | |
| | 0.0066 M (approx. 0.1%) | | | | 0.066 M (approx. 1%) | | | |
| | | PVP | PVP | PVP | | PVP | PVP | PVP |
| | | K17 | K17 | K30 | | K17 | K17 | K30 |
| | as is | 0.5% | 2% | 2% | as is | 0.5% | 2% | 2% |
| Time zero | 6.34 | 6.39 | 6.39 | 6.45 | 6.29 | 6.34 | 6.35 | 6.37 |
| 65° C. | | | | | | | | |
| 10 days | 5.03 | 6.23 | 6.14 | 5.89 | 6.16 | 6.32 | 6.34 | 6.29 |
| 15 days | 4.24 | 6.06 | 5.86 | 5.47 | 6.05 | 6.19 | 6.25 | 6.19 |
| 1 month | 3.94 | 6.13 | 5.00 | 4.82 | 6.13 | 6.21 | 6.31 | 6.21 |
| 40° C. | | | | | | | | |
| 3 months | 5.16 | 6.19 | 6.05 | 5.75 | 6.09 | 6.12 | 6.14 | 6.11 |
| 6 months | 3.82 | 6.14 | 5.44 | 5.29 | 6.11 | 6.19 | 6.24 | 6.20 |

It is evident that when concentrations of conventional buffering agents are present in the formulation (as in the case of phosphate buffer in the unusual low concentration of about 0.1%), PVP K17 and PVP K30 exert a synergistic effect in controlling the pH value, as clearly shown in particular in the case of batch 13169/12-E, where the superior properties of PVP K17 in comparison with PVP K30 are highlighted. When conventional buffering agents are used in usual concentrations, their buffering capacity is sufficient per se, but, as it is shown in the following example 3 with detrimental effects on the physical stability of the formulations.

### EXAMPLE 3

The detrimental effect of significant concentration of buffering agents, such as phosphate buffer, on the physical performances of steroidal drug aqueous suspension is shown in the following tables 3 and 4.

The tested suspension basic composition is the following: Medroxyprogesterone acetate 20%, MyristylGammaPicoliniumChloride 0.2%, sodium sulphate 1. 1%, PEG 3350 2.03%, Sodium hydroxide q.s. to pH 6.5 (*), WFI q.s to 100 ml. polyvinylpyrrolidone and phosphate buffer were added according the scheme in Table 3 and 4. (*) The pH was adjusted after adding PVP but not after adding buffer.

The polyvinylpyrrolidone used was a Kollidon K17PF from BASF.

Filling volume—Primary packaging information: 0.75 ml suspension/ vial (upright)—3 ml clear type I siliconized glass vials, 13 mm diameter Omniflex® rubber stoppers, aluminum seals.

The phosphate buffer 0.0066 M (approximately 0.1%) has the following composition: Monobasic sodium phosphate hydrate 69.4 mg and dibasic sodium phosphate dodecahydrate 58.8 mg in 100 mL of suspension; while the phosphate buffer 0.066 M (approximately 1%) has the following composition: Monobasic sodium phosphate hydrate 694 mg and dibasic sodium phosphate dodecahydrate 588 mg in 100 mL of suspension.

TABLE 3

| | 13451/01 - 1 (as is) | | | | | |
|---|---|---|---|---|---|---|
| | | | B: Phosphate | | C: Phosphate | |
| | | | Buffer | | Buffer | |
| | A: as is | | Approx. 0.1% | | Approx. 1% | |
| | Res. | Syr. | Res. | Syr. | Res. | Syr. |
| Time | R = 20 | MT | R = 25 | MT | R = 24 | MT |
| zero | T = 7 s | | T = 8 s | | T = 10 s | |
| 55° C. | R = 50 | MT | R = >50 | MT | R = >50 | MT |
| 1 m | T = 18 s | | T = 29 s | | T = 40 s | |
| 55° C. | R = >50 | MT | R = >50 | MT | Not | NA |
| 2 m | T = 33 s | | T = 24 s | | resuspendable | |
| 55° C. | R = >50 | MT | R = >50 | MT | Not | NA |
| 3 m | T = 31 s | | T = 32 s | | resuspendable | |

Res. = Resuspendability
T = time of manual wrist shaking requested in order to obtain a homogeneous suspension (seconds),
R = number of revolutions requested in order to obtain a homogeneous suspension
Syr. = Syringeability 3 vials, needle 26G × 1/2″;
MT = meets test;
DNMT = does not meet test
NA = not applicable as the product cannot be resuspended

TABLE 4

| | 13451/01 - 2 (+PVP K17 2%) | | | | | |
|---|---|---|---|---|---|---|
| | | | B: Phosphate | | C: Phosphate | |
| | | | Buffer | | Buffer | |
| | A: as is | | Approx. 0.1% | | Approx. 1% | |
| | Res. | Syr. | Res. | Syr. | Res. | Syr. |
| Time | R = 26 | MT | R = 27 | MT | R = 27 | MT |
| zero | T = 7 s | | T = 5 s | | T = 8 s | |
| 55° C. | R = 45 | MT | R = 50 | MT | Not | NA |
| 1 m | T = 10 s | | T = 17 s | | resuspendable | |
| 55° C. | R = >50 | MT | R = >50 | MT | Not | NA |
| 2 m | T = 27 s | | T = 34 s | | resuspendable | |
| 55° C. | R = >50 | MT | R = >50 | MT | Not | NA |
| 3 m | T = 31 s | | T = 35 s | | resuspendable | |

Res. = Resuspendability
T = time of manual wrist shaking requested in order to obtain a homogeneous suspension (seconds),
R = number of revolutions requested in order to obtain a homogeneous suspension
Syr. = Syringeability 3 vials, needle 26G × 1/2″;
MT = meets test;
DNMT = does not meet test
NA = not applicable as the product cannot be resuspended

As clearly shown in the above tables, the usual concentration of an inorganic acid salt as buffering agent has a negative and significant effect on the suspension re-suspendability and syringeability. On the contrary, a lower and unusual concentration of an inorganic acid salt as buffering agent does not negatively affect the physical performances of the formulation. As shown in example 4, when such a low concentration of buffer is used in combination with PVP, a relevant pH controlling effect is obtained.

### EXAMPLE 4

The influence of PVP K17PF as pH controlling agent on a prototype formulation of medroxyprogesterone acetate is shown in Table 5, where the measured pH values recovered in an accelerated stability study are shown.

The tested suspension basic composition is the following: Medroxyprogesterone acetate 20%, MyristylGammaPicoliniumChloride 0.2%, Sodium sulphate 1.1%, PEG 3350 2.03%, Sodium hydroxide q.s. to pH 6.5 (*), WFI q.s to 100 ml. Polyvinylpyrrolidone and phosphate buffer are added according to the scheme in Table 5. (*) The pH was adjusted after adding PVP but not after adding buffer.

The polyvinylpyrrolidone used was a Kollidon K17PF from BASF.

Filling volume—Primary packaging information: 0.75 ml suspension/vial (upright)—3 ml clear type I siliconized glass vials, 13 mm diameter Omniflex® rubber stoppers, aluminum seals.

The phosphate buffer 0.0066 M (approximately 0.1%) has the following composition: Monobasic sodium phosphate hydrate 69.4 mg and dibasic sodium phosphate dodecahydrate 58.8 mg in 100 mL of suspension.

TABLE 5

| | | | 13451/01-2 | |
|---|---|---|---|---|
| | 13451/01-1 (as is) | | (+PVP K17 2%) | |
| | | B: Phosphate | | B: Phosphate |
| 13451/01 | A: as is | Buffer 0.1% | A: as is | Buffer 0.1% |
| Time zero | 6.35 | 6.71 | 6.28 | 6.74 |
| 65° C. - 10 days | 3.20 | 4.53 | 3.46 | 5.72 |
| 15 days | 3.12 | 3.60 | 3.28 | 4.30 |
| 1 month | 2.95 | 3.32 | 3.08 | 3.58 |
| 55° C. - 1 month | 3.12 | 3.67 | 3.24 | 6.16 |
| 2 months | 2.92 | 3.28 | 2.97 | 4.77 |
| 3 months | 2.83 | 3.15 | 2.97 | 3.81 |
| 40° C. - 3 months | 2.93 | 3.67 | 3.24 | 6.19 |

It is evident that when reduced concentrations of conventional buffering agents are present in the formulation (as in the case of Phosphate buffer concentrations of about 0.1%), PVP K17PF exerts a synergistic effect in controlling the pH value.

### EXAMPLE 5

The effective control obtained on the pH of a suspension formulation of an association of medroxyprogesterone acetate and estradiol cypionate containing a combination of PVP K17PF, a phosphate buffer 0.0066 M (approximately 0.1%) is shown in Table 7, where the measured pH values recovered in an accelerated stability study are shown.

The tested suspension composition (batch 13169/57) is the following: Medroxyprogesterone acetate 5%, estradiol cypionate 1%, methylparaben 0.18%, propylparaben 0.02%, sodium chloride 0.856%, PEG 3350 2.856%, polysorbate 80 0.19%, sodium hydroxide q.s. to pH 6.5, WFI q.s to 100 ml. polyvinylpyrrolidone and phosphate buffer are added according to the scheme in Table 7. The pH is adjusted after adding PVP but not after adding buffer.

The polyvinylpyrrolidone used was a Kollidon K17PF from BASF. Filling volume—Primary packaging information: 20 ml suspension/ vial (upright)—20/26 ml clear type I siliconized glass vials, 20 mm diameter Helvoinert® rubber stoppers, aluminum seals.

The phosphate buffer 0.0066 M (approximately 0.1%) has the following composition: Monobasic sodium phosphate hydrate 69.4 mg and dibasic sodium phosphate dodecahydrate 58.8 mg in 100 mL of suspension.

TABLE 7

| | | | 13169/57-2B |
|---|---|---|---|
| | | | (+PVP K17 2% + |
| | 13169/57-1 | 13169/57-2A | Phosphate |
| | (as is) | (+PVP K17 2%) | Buffer ≃0.1% |
| Time zero | 6.48 | 6.49 | 6.65 |
| 65° C. - 10 days | 5.48 | 5.23 | 6.50 |
| 65° C. - 15 days | 3.79 | 4.76 | 6.31 |
| 65° C. - 1 month | 3.25 | 4.51 | 6.17 |
| 65° C. - 2 months | 3.12 | 4.32 | 5.88 |
| 55° C. - 1 month | 3.47 | 4.79 | 6.34 |
| 55° C. - 2 months | 3.06 | 4.56 | 6.22 |
| 55° C. - 3 months | 3.03 | 4.49 | 6.12 |

As evident PVP K17PF either used alone or in combination with a lower and unusual concentration of phosphate buffer is able to exert a pH controlling effect on the suspension formulation.

The following are examples of pharmaceutical compositions according to the invention and are not intended to limit the scope of the invention itself.

### EXAMPLE A

Stabilized Parenteral Aqueous suspension of Medroxy
Progesterone Acetate.
The formulation is as follows (% w/v):

| | |
|---|---|
| Medroxyprogesterone Acetate (micronized) | 20% |
| Myristyl Gamma Picolinium Chloride | 0.2% |
| Sodium Sulphate | 1.1% |
| Polyethylene Glycol 3350 | 2.03% |
| Polyvinylpyrrolidone K17 | 2.0% |
| Monobasic Sodium Phosphate hydrate | 0.0694% |
| Dibasic Sodium Phosphate dodecahydrate | 0.0588% |
| Sodium Hydroxide or Hydrochloric Acid q.s. to | PH 6.0-7.0 |
| Water for Injections q.s. to | 100 ml |

The excipients are dissolved in Water for Injections. The obtained vehicle is sterilized by steam sterilization or sterilant filtration. Sterile micronized medroxprogesterone acetate is added to the vehicle, the obtained suspension is passed through a suitable homogenizer in aseptic condition and the pH is adjusted. The homogeneous suspension is then aseptically distributed in single-use containers.

The obtained product has desirable properties for parenteral use, keeps well and has a substantially stabilized pH.

### EXAMPLE B

Stabilized Parenteral Aqueous Suspension of Medroxy
Progesterone Acetate.
The formulation is as follows (% w/v):

| | |
|---|---|
| Medroxyprogesterone Acetate | 16% |
| Methylparaben | 0.16% |
| Propylparaben | 0.015% |
| Sodium Chloride | 0.8% |
| Polyethylene Glycol 3350 | 2.875% |
| Polysorbate 80 | 0.3% |
| Polyvinylpyrrolidone K17 | 0.5% |
| Monobasic Sodium Phosphate hydrate | 0.0694% |
| Dibasic Sodium Phosphate dodecahydrate | 0.0588% |
| Sodium Hydroxide or Hydrochroric Acid q.s. to | PH 6.0-7.0 |
| Water for Injections q.s. to | 100 ml |

The manufacturing method include preparation of a sterile vehicle, aseptic compounding of sterile micronized medroxyprogesterone acetate into the vehicle and aseptic distribution of the obtained sterile homogenous suspension into single dose container.

The product has a substantially stabilized pH, good resuspendability and can be administered with a syringe-needle suitable for subcutaneous and intramuscular use.

### EXAMPLE C

Stabilized Parenteral Aqueous Suspension of a
combination of Medroxyprogesterone
Acetate and Estradyol Cypionate.
The formulation is as follows (% w/v):

| | |
|---|---|
| Medroxyprogesterone Acetate (micronized) | 5% |
| Estradiol Cypionate (micronized) | 1% |
| Methylparaben | 0.180% |
| Propylparaben | 0.020% |
| Sodium Chloride | 0.856% |
| Polyethylene Glycol 3350 | 2.856% |
| Polysorbate 80 | 0.190% |
| Polyvinylpyrrolidone K17 | 2.000% |
| Monobasic Sodium Phosphate hydrate | 0.0694% |
| Dibasic Sodium Phosphate dodecahydrate | 0.0588% |
| Sodium Hydroxide or Hydrochloric Acid q.s. to | pH 6.0-7.0 |
| Water for Injections q.s. to | 100 ml |

The parabens are dissolved in Water for Injections previously heated at approximately 70-90° C. The parabens solution is cooled down to room temperature, the remaining excipients are added and dissolved and the pH is adjusted to the desired range.

Micronized medroxyprogesterone acetate and estradiol cypionate are slurried into the vehicle and the obtained dispersion is homogenized until a fine, syringeable suspension is obtained.

In order to obtain a sterile suspension suitable for parenteral administration sterile active drugs and vehicle are used and the obtained suspension aseptically distributed into single dose containers.

The obtained product can be easily resuspended and can easily flow though a syringe needle, has a substantially stabilized pH and is suitable for intradermal, subcutaneous and intramuscular administration.

### EXAMPLE D

Stabilized Parenteral Aqueous Suspension of Exemestane.
The formulation is as follows (% w/v):

| | |
|---|---|
| Exemestane (micronized) | 10% |
| Methylparaben | 0.16% |
| Propylparaben | 0.015% |
| Sodium Chloride | 0.9% |
| Polyethylene Glycol 4000 | 3.0% |
| Polysorbate 80 | 0.3% |
| Polyvinylpyrrolidone K17 | 2.0% |
| Monobasic Sodium Phosphate hydrate | 0.0694% |
| Dibasic Sodium Phosphate dodecahydrate | 0.0588% |
| Sodium Hydroxide or Hydrochloric Acid q.s. to | pH 6.0-7.0 |
| Water for Injections q.s. to | 100 ml |

The excipients are dissolved in Water for Injections and the obtained solution sterilized by sterilant filtration. The pH is adjusted and sterile exemestane is added.

The obtained suspension is passed through a suitable homogenizer until a fine, syringeable suspension is obtained and then aseptically distributed.

The product has desirable properties for parenteral use, keeps well and has a substantially stabilized pH.

1. A pharmaceutical aqueous suspension formulation for parenteral administration having substantially stabilized pH, comprising a biologically active compound and a pH controlling effective concentration of a polyvinylpyrrolidone compound.

2. A pharmaceutical formulation, according to claim 1, wherein the pH controlling effective concentration of a polyvinylpyrrolidone compound is from about 0.1% w/v or w/w to about 10% w/v or w/w.

3. A pharmaceutical formulation, according to claim 2, wherein the molecular weight of the polyvinylpyrrolidone compound is from about 7000 to about 54000.

4. A pharmaceutical formulation, according to claim 3, wherein the molecular weight of the polyvinylpyrrolidone compound is from about 7000 to about 1000.

5. A pharmaceutical composition, according to claim 4, wherein the pH controlling effective amount of a polyvinylpyrrolidone compound is from about 0.2% w/w or w/v to about 5% w/w or w/v.

6. A pharmaceutical aqueous suspension formulation for parenteral administration having substantially stabilized pH, comprising a biologically active compound, a buffering agent and a polyvinylpyrrolidone compound in concentrations effective to produce a pH controlling superadditive effect.

7. A pharmaceutical composition, according to claim 6, wherein the buffering agent is a phosphoric acid salt in a concentration lower than 0.4% w/v or w/w.

8. A pharmaceutical composition, according to claim 7, wherein the concentration of the phosphoric acid salt is lower than 0.2% w/v or w/w.

9. A pharmaceutical composition, according to any preceding claims, wherein the pH range of formulation is from about pH 3.0 to about pH 8.0.

10. A pharmaceutical composition, according to claim 9, wherein the biologically active compound is a steroidal compound.

11. A pharmaceutical composition, according to claim 10, wherein the biologically active steroidal compound is selected from exemestane, medroxyprogesterone acetate and estradiol cypionate or a combination of medroxyprogesterone acetate and estradiol cypionate.

12. Use of a polyvinylpyrrolidone compound, in the preparation of a pharmaceutical aqueous suspension formulation having substantially stabilized pH, for parenteral administration of a biologically active compound, characterized in that a pH controlling effective concentration of polyvinylpyrrolidone is added thereto.

13. Use of a polyvinylpyrrolidone compound and a buffering agent in concentrations effective to produce a pH controlling superadditive effect, in the preparation of a pharmaceutical aqueous suspension formulation having substantially stabilized pH, for parenteral administration of a biologically active compound.
